(19) United States
(12) Patent Application Publication
(10) Pub. No.: US 2024/0278024 A1
(43) Pub. Date: Aug. 22, 2024

(54) BORE STACKING TOLERANCE

(71) Applicant: Cardiac Pacemakers, Inc., St. Paul, MN (US)

(72) Inventors: Robert Allen Jones, Lake Elmo, MN (US); James Michael English, Cahir (IE)

(21) Appl. No.: 18/439,573

(22) Filed: Feb. 12, 2024

Related U.S. Application Data
(60) Provisional application No. 63/446,929, filed on Feb. 20, 2023.

(51) Int. Cl. A61N 1/375

(52) U.S. Cl. CPC A61N 1/37512; A61N 1/3754; A61N 1/3758

(57) ABSTRACT

Systems and methods are disclosed to absorb a portion of a positive or negative manufacturing tolerance of at least one component of a stacked bore assembly are disclosed, the stacked bore assembly comprising a first component having a distal mechanical feature and a second component having a proximal mechanical feature configured to engage the distal mechanical feature the first component to a shoulder on a first one of the distal mechanical feature of the first component or the proximal mechanical feature of the second component. The shoulder can be positioned at a distance from an end of the first one of the distal mechanical feature of the first component or the proximal mechanical feature of the second component shorter or longer than a length of a second of the distal mechanical feature of the first component or the proximal mechanical feature of the second component.

### CLAIM OF PRIORITY

This application claims the benefit of U.S. Provisional Application No. 63/446,929 filed on Feb. 20, 2023, which is hereby incorporated by reference in its entirety.

### TECHNICAL FIELD

This document relates generally to medical devices and more particularly to systems and methods to ensure tolerances in stacked bore assemblies.

### BACKGROUND

Medical devices can be implanted or implantable in a body of a patient, such as to monitor patients, including detecting or sensing physiologic information from the patient, such as one or more of heart sounds, respiration (e.g., respiratory rate (RR), tidal volume (TV), etc.), impedance (e.g., thoracic impedance, cardiac impedance, cutaneous impedance, etc.), pressure (e.g., blood pressure), cardiac activity (e.g., heart rate, cardiac electrical information, etc.), chemical (e.g., electrolyte), physical activity, posture, plethysmography, or one or more other physiologic information of the patient, and, in certain examples, provide therapy to the patient in clinical and ambulatory settings. Implantable medical devices (IMDs) can include cardiac rhythm management (CRM) devices, such as pacemakers, cardiac resynchronization devices, cardioverters, defibrillators, drug delivery devices, or one or more other medical devices implanted or implantable within a body of, or subcutaneously to, a patient.

Implantable medical devices often include a hermetically sealed housing containing electronic circuitry of the implantable medical device (e.g., one or more signal processing or control circuits, telemetry circuits, therapy circuits, power management circuits, etc.) and a power source, and one or more bore assemblies (or lead ports or lead connector cavities, etc.) in a header outside of the hermetically sealed housing to receive and couple one or more leads having one or more electrodes or other sensors positioned at various locations in or near a heart of the patient, such as in one or more of the atria or ventricles, to the implantable medical device. Separate from, or in addition to, the one or more electrodes or other sensors of the leads, the implantable medical device can include one or more electrodes or other sensors (e.g., a pressure sensor, an accelerometer, a gyroscope, a microphone, etc.) powered by a power source in the implantable medical device. The one or more electrodes or other sensors of the leads, the implantable medical device, or a combination thereof, can be configured detect physiologic information from the patient, or provide one or more therapies or stimulation to the patient.

Bore assemblies include various numbers and configurations of electrical contacts for communicating electrical signals into and out of the implantable medical device, such as between the electronic circuitry of the implantable medical device and one or more electrodes coupled to the one or more leads, for example, depending on the type of medical device, the function being performed by the medical device, and the specific lead or leads to be coupled thereto. In certain examples, an individual bore assembly can include multiple individual components stacked and connected in various configurations to form a number of different stacked bores having different desired physical and electrical configurations. The present inventors have recognized, among other things, a need to reduce stack up tolerance in different combinations of multi-component stacked bores to reduce cost and complexity of manufacture and assembly of medical device bore assemblies, medical device headers, and medical devices in general.

### SUMMARY

Systems and methods are disclosed to absorb a portion of a positive or negative manufacturing tolerance of at least one component of a stacked bore assembly are disclosed, the stacked bore assembly comprising a first component having a distal mechanical feature and a second component having a proximal mechanical feature configured to engage the distal mechanical feature the first component to a shoulder on a first one of the distal mechanical feature of the first component or the proximal mechanical feature of the second component. The shoulder can be positioned at a distance from an end of the first one of the distal mechanical feature of the first component or the proximal mechanical feature of the second component shorter or longer than a length of a second of the distal mechanical feature of the first component or the proximal mechanical feature of the second component.

The stacked bore assembly can include multiple components assembled to receive a lead in a bore of the stacked bore assembly and to electrically couple to one or more electrical contacts on the lead once the lead is inserted into and secured in the bore of the stacked bore assembly. In an example, the distal mechanical feature of the first component can be configured to engage the proximal mechanical feature of the second component to a shoulder on the proximal mechanical feature of the second component, wherein the shoulder is positioned at a distance from a proximal end of the second component longer than a length of the distal mechanical feature of the first component to absorb a portion of one of a positive or negative manufacturing tolerance of at least one of the first or second components. In another example, the distal mechanical feature of the first component can be configured to engage the proximal mechanical feature of the second component to a shoulder on the proximal mechanical feature of the second component, wherein the shoulder is positioned at a distance from a proximal end of the second component longer than a length of the distal mechanical feature of the first component to absorb a portion of one of a positive or negative manufacturing tolerance of at least one of the first or second components.

An example (e.g., “Example 1”) of subject matter (e.g., a system) may comprise a stacked bore assembly comprising multiple components between proximal and distal ends of the stacked bore assembly, the stacked bore assembly configured to receive a lead in a bore of the stacked bore assembly and to electrically couple to one or more electrical contacts on the lead once the lead is inserted into and secured in the bore of the stacked bore assembly, the stacked bore assembly comprising: a first component comprising a distal mechanical feature and a second component comprising a proximal mechanical feature configured to engage the distal mechanical feature of the first component to a shoulder on a first one of the distal mechanical feature of the first component or the proximal mechanical feature of the second component, wherein the shoulder is positioned at a distance from an end of the first one of the distal mechanical feature of the first component or the proximal mechanical feature of the second component shorter or longer than a length of a second of the distal mechanical feature of the first component or the proximal mechanical feature of the second component to absorb a portion of a positive or negative manufacturing tolerance of at least one of the first component or the second component.

In Example 2, the subject matter of Example 1 may optionally be configured such that the stacked bore assembly is a component of a header of an implantable medical device, the implantable medical device comprises a housing comprising electronic circuitry, the first component comprises a first electrical contact to couple a first electronic circuit of the electronic circuitry of the housing of the implantable medical device to the first electrical contact of the stacked bore assembly, the second component comprises a second electrical contact to couple a second electronic circuit of the electronic circuitry of the housing of the implantable medical device to the second electrical contact of the stacked bore assembly, the stacked bore assembly is configured to receive a lead comprising first and second electrical contacts, the first electrical contact of the lead, when inserted into and retained by the stacked bore assembly, is configured to couple to the first electrical contact of the first component of the stacked bore assembly, and the second electrical contact of the lead, when inserted into and retained by the stacked bore assembly, is configured to couple to the second electrical contact of the second component of the stacked bore assembly.

In Example 3, the subject matter of any one or more of Examples 1-2 may optionally be configured such that the distal mechanical feature of the first component comprises a male mechanical feature and the proximal mechanical feature of the second component comprises a female mechanical feature configured to interference fit with the male mechanical feature to the shoulder on one of the male mechanical feature or the female mechanical feature.

In Example 4, the subject matter of any one or more of Examples 1-3 may optionally be configured such the distal mechanical feature of the first component comprises a female mechanical feature and the proximal mechanical feature of the second component comprises a male mechanical feature configured to interference fit with the female mechanical feature to the shoulder on one of the male mechanical feature or the female mechanical feature.

In Example 5, the subject matter of any one or more of Examples 1˜4 may optionally be configured such that the second component comprises a distal mechanical feature and the stacked bore assembly comprises a third component comprising a coupler having proximal and distal mechanical features and a fourth component comprising a proximal mechanical feature, wherein the coupler is configured to engage the distal mechanical feature of the second component and the proximal mechanical feature of the fourth component between respective shoulders of the second component and the fourth component, the coupler having a length shorter or longer than a corresponding distance between the respective shoulders when the fourth component is joined with the second component by the third component to absorb a portion of one of a positive or negative manufacturing tolerance of at least one of the second component, the third component, or the fourth component.

In Example 6, the subject matter of any one or more of Examples 1-5 may optionally be configured such that the coupler comprises a conductive material configured to be electrically connected to a second electrical contact of the second component when the coupler is engaged with the second component, the second component comprises a core component composed of an insulator material including an interior mechanical feature to position the second electrical contact of the second component with an electrical contact of the lead when inserted into and retained by the stacked bore assembly, wherein the insulator material of the second component is configured to electrically insulate the conductive material of the coupler from a first electrical contact of the first component, and the fourth component comprises a core component substantially similar to the second component.

In Example 7, the subject matter of any one or more of Examples 1-6 may optionally be configured such that the first component comprises a tip component at a proximal end of the stacked bore assembly, the tip component configured to receive and retain a proximal end of a lead once inserted into the stacked bore assembly and the stacked bore assembly comprises multiple core components joined by multiple couplers between the tip component and a distal end of the stacked bore assembly.

In Example 8, the subject matter of any one or more of Examples 1-7 may optionally be configured such that the distal mechanical feature of the second component comprises a transition of mechanical fits with respect to a proximal end of the third component, from a slip or transition fit with the proximal mechanical feature of the third component when the third component is engaged with the second component at a distal end of the distal mechanical feature to an interference fit at a proximal end of the distal mechanical feature, wherein the length of the interference fit is greater than a positive or negative manufacturing tolerance of at least one of the second component or the third component.

In Example 9, the subject matter of any one or more of Examples 1-8 may optionally be configured such that the shoulder is positioned at the distance from an edge of the distal mechanical feature of the first component shorter than the length of the proximal mechanical feature of the second component to absorb a portion of the positive manufacturing tolerance of at least one of the first component or the second component.

In Example 10, the subject matter of any one or more of Examples 1-9 may optionally be configured such that the shoulder includes a first interior shoulder on a female mating component of a distal end of the first component and a second exterior shoulder on a male mating component of a proximal end of the second component, wherein the first interior shoulder on the female mating component of the distal end of the first component is positioned at a distance shorter than a length of the male mating component of the proximal end of the second component to absorb a portion of a positive or negative manufacturing tolerance of at least one of the first component or the second component and the length of the male mating component of the proximal end of the second component comprises a distance from the proximal end of the second component to the second exterior shoulder on the male mating component of the proximal end of the second component.

In Example 11, the subject matter of any one or more of Examples 1-10 may optionally be configured such that the shoulder includes a first interior shoulder on a female mating component of a distal end of the first component and a second exterior shoulder on a male mating component of a proximal end of the second component, wherein the second exterior shoulder on the male mating component of the proximal end of the second component is positioned at a distance longer than a length of the female mating component of the distal end of the first component from the proximal end of the second component to absorb a portion of a positive or negative manufacturing tolerance of at least one of the first component or the second component and the length of the female mating component of the distal end of the first component comprises a distance from the distal end of the first component to the first interior shoulder on the female mating component of the distal end of the first component.

In Example 12, the subject matter of any one or more of Examples 1-11 may optionally be configured such that the distance of the position of the second exterior shoulder from proximal end of the second component comprises an aggregate positive or negative manufacturing tolerance of at least one first or second components with respect to a plane defined by one of the first component or the second component.

In Example 13, the subject matter of any one or more of Examples 1-12 may optionally be configured such that the distance of the position of the second exterior shoulder from proximal end of the second component comprises the positive or negative manufacturing tolerance of at least one the female mating component of the distal end of the first component or the male mating component of the proximal end of the second component.

An example (e.g., “Example 14”) of subject matter (e.g., a system) may comprise a stacked bore assembly comprising multiple components, the stacked bore assembly configured to receive a lead in a bore of the stacked bore assembly and to electrically couple to one or more electrical contacts on the lead once the lead is inserted into and secured in the bore of the stacked bore assembly, the stacked bore assembly comprising a first component comprising a distal mechanical feature and a second component comprising a proximal mechanical feature configured to engage the distal mechanical feature of the first component, wherein the distal mechanical feature of the first component is configured to engage the proximal mechanical feature of the second component to a shoulder on the proximal mechanical feature of the second component and the shoulder is positioned at a distance from a proximal end of the second component longer than a length of the distal mechanical feature of the first component to absorb a portion of one of a positive or negative manufacturing tolerance of at least one of the first component or the second component.

In Example 15, the subject matter of any one or more of Examples 1-14 may optionally be configured such that the second component comprises a distal mechanical feature and the stacked bore assembly comprises a third component comprising a coupler to engage a proximal mechanical feature of a fourth component and the distal mechanical feature of the second component between respective shoulders of the second component and the fourth component, the coupler having a length shorter or longer than a corresponding distance between the respective shoulders when the fourth component is joined with the second component by the third component to absorb a portion of one of a positive or negative manufacturing tolerance of at least one of the second component, the third component, or the fourth component.

In Example 16, the subject matter of any one or more of Examples 1-15 may optionally be configured such that the first component and the third component include conductive material to couple to respective feedthrough connections of a housing of an implantable medical device and the second component includes an insulator to insulate the conductive material of the first component from the conductive material of the third component.

In Example 17, the subject matter of any one or more of Examples 1-16 may optionally be configured such that the first component comprises a tip component of the stacked bore assembly, the second component and the fourth component comprise core components of the stacked bore assembly, the third component comprises a coupler, and the stacked bore assembly comprises multiple core components and multiple couplers between proximal and distal ends of the stacked bore assembly, each coupler and core component substantially similar to the other respective couplers or core components.

In Example 18, the subject matter of any one or more of Examples 1-17 may optionally be configured to include a strain relief component at a distal end of the stacked bore assembly, opposite the tip component.

An example (e.g., “Example 19”) of subject matter (e.g., a system) may comprise a stacked bore assembly comprising multiple components, the stacked bore assembly configured to receive a lead in a bore of the stacked bore assembly and to electrically couple to one or more electrical contacts on the lead once the lead is inserted into and secured in the bore of the stacked bore assembly, the stacked bore assembly comprising a first component comprising a distal mechanical feature and a second component comprising a proximal mechanical feature configured to engage the distal mechanical feature of the first component to a shoulder on the distal mechanical feature of the first component, wherein the shoulder is positioned at a distance from a distal end of the first component longer than a length of the proximal mechanical feature of the second component to absorb a portion of one of a positive or negative manufacturing tolerance of at least one of the first component or the second component.

In Example 20, the subject matter of any one or more of Examples 1-19 may optionally be configured such that the second component comprises a distal mechanical feature, the stacked bore assembly comprises a third component comprising a coupler having proximal and distal mechanical features and a fourth component comprising a proximal mechanical feature, the coupler is configured to engage the distal mechanical feature of the second component and the proximal mechanical feature of the fourth component between respective shoulders of the second component and the fourth component, the coupler having a length shorter or longer than a corresponding distance between the respective shoulders when the fourth component is joined with the second component by the third component to absorb a portion of one of a positive or negative manufacturing tolerance of at least one of the second component, the third component, or the fourth component, and the distal mechanical feature of the second component comprises a transition of mechanical fits with respect to a proximal end of the third component, from a slip or transition fit with the proximal mechanical feature of the third component when the third component is engaged with the second component at a distal end of the distal mechanical feature to an interference fit at a proximal end of the distal mechanical feature, wherein the length of the interference fit is greater than a positive or negative manufacturing tolerance of at least one of the second component or the third component.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example cross section of a stacked bore assembly for placement in a header of an implantable medical device.

FIGS. 2-3 illustrate different subsets of components of the stacked bore assembly.

FIG. 4 illustrates an example implantable medical device comprising the stacked bore assembly.

FIG. 5 illustrates an example medical device system.

FIG. 6 illustrates an example patient management system and portions of an environment in which the patient management system may operate.

FIG. 7 illustrates an example method of assembling a stacked bore assembly and subsequently assembling an implantable medical device.

FIG. 8 illustrates a block diagram of an example machine upon which any one or more of the techniques (e.g., methodologies) discussed herein may perform.

### DETAILED DESCRIPTION

Traditional lead bores (or lead ports or lead connector cavities, etc.) include respective, separate lead bores designed and configured for different leads and respective combinations of leads, electrical contacts, and connections for different implantable medical devices. To service medical devices configured for different leads or combinations of lead bores, medical device manufacturers design, manufacture, stock, and service a number of different traditional lead bores for each respective lead or combination of lead bores, at substantial cost.

In contrast to traditional lead bores, multi-element stacked bore assemblies (or stacked lead port assemblies or stacked lead connector cavities, etc.) have developed, combining different components to create various permutations of respective combinations of leads and implantable medical devices, reducing the amount of finished product that must be stocked for each combination, and enabling re-use of individual components in the different combinations. Stacked bore assemblies, in contrast to traditional lead bores, can reduce the overall manufacture cost of implantable medical devices having different permutations of leads or combinations of leads while increasing the number of leads compatible with the implantable medical device.

However, each component in the multi-component stacked bore assemblies separately have respective manufacturing tolerances. The combination of the separate respective tolerances can form an aggregate stack up tolerance greater than traditional lead bores that can bring the stacked bore assembly out of spec, requiring additional assembly steps or procedures, or higher tolerance manufacturing steps, reducing the benefit of the stacked bore assembly. The present inventors have recognized, among other things, that tolerances of individual components can be biased to one side of the assembly, in certain examples, leaving float or free space to reduce aggregate stack up tolerance and maintain assembled configurations within spec. Traditional tolerance is typically ±an amount, such as ±5 thou ( 5/1000 of an inch), etc. Although described herein as the same amount plus as minus, in certain examples tolerances can be plus and minus different amounts. In certain examples, biasing tolerances in the physical design of individual components, such as by adding strategic clearances to one or more components, can absorb one side of the traditional tolerance in a component or between two components, such that instead of ±5 thou, the resulting tolerance in the biased combination can be between 0 and 5 thou (or between −5 thou and 0, depending on orientation), such as by adding 5 thou of free space or clearance in the design of one component to absorb positive tolerance (or negative tolerance) in one or more other components, such as between two interference fit components, such as shown and described herein.

Such free space or clearance between components can be advantageous over other methods of reducing stack up tolerance, such as increased precision in manufacturing processes, custom sized spacers, or compressible seals. For example, increased precision and custom sized spacers add otherwise unnecessary manufacturing costs and intervening steps, including measurement. Compressible seals add additional tolerances of their own, require force, break down over time, are not linearly pliable during compression, and exert a negative force pushing components away under compression.

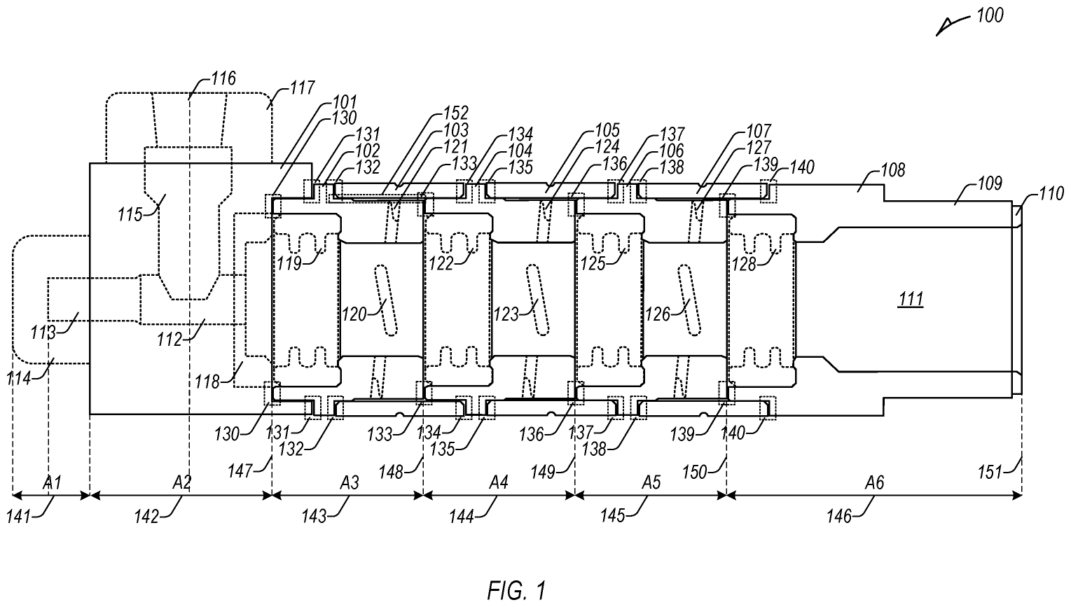

FIG. 1 illustrates an example cross section of a stacked bore assembly 100 for placement in a header of an implantable medical device. The stacked bore assembly 100 includes multiple components that, when mechanically coupled, such as by aligning and fitting together the multiple components with a slip, push, or compression fit between male and female mechanical joining features, form a bore to receive and secure a proximal end of a lead and to electrically couple to one or more electrical contacts on the lead once the lead is inserted into and secured in the bore of the stacked bore assembly 100.

The stacked bore assembly 100 includes a proximal end (at left in FIG. 1) and a distal end (at right in FIG. 1) comprising multiple components including a first component 101 (e.g., a tip component) at the proximal end coupled to a second component 102 (e.g., a core component) distal to the first component 101, the second component 102 coupled to a third component 103 (e.g., a coupler) distal to the second component 102, proceeding in this example with additional components (e.g., subsequent combinations of core components and couplers, such as fourth through seventh components 104-107) until an eighth component 108 (e.g., a strain relief component) at the distal end of the stacked bore assembly 100 including a bore opening 111 of a bore that continues back through the multiple components (e.g., the one or more core components) of the stacked bore assembly 100 to the first component 101 (e.g., the tip component).

Different components of the stacked bore assembly 100 can include electrical contacts, or be composed of a conductive material, separated by one or more insulators, configured to enable coupling of one or more electrical contacts of a coupled lead to one or more other contacts of a medical device comprising the stacked bore assembly 100. For example, the stacked bore assembly 100 can be a component of a header of an implantable medical device and can be configured to provide an electrical connection between the one or more electrical contacts of a lead to one or more electrical contacts or electronic circuits in the implantable medical device, such as through one or more feedthrough pins, wires, or combinations thereof, etc.

The first component 101 (e.g., the tip component) can include different features to receive and retain a proximal end of a lead, including a bore end 113, an end cap 114 (e.g., a translucent end cap to confirm full insertion of the lead in the stacked bore assembly 100), a bore retention portion 112 coupled to a retention channel 115 comprising a mechanical retention feature (e.g., a set screw or fastener) available through an opening 116 in a collar 117 to secure the lead in the stacked bore assembly 100, and a seat 118 to receive a mechanical feature (e.g., a shoulder) of the lead. In certain examples, the first component 101 can be composed of a conductive material or can include a conductive portion to electrically couple to an electrical contact of the lead. In other examples, the first component 101 be composed of an electrically insulative material and optionally include one or more conductive portions or conductive material, such as to contact an electrical contact of the lead, to provide a mating surface for a conductor coupled to a feedthrough of the implantable medical device, etc.

The second component 102 (e.g., a core component) can be composed of an electrically insulative material, such as to insulate one or more other conductors or conductive elements from the first component 101 (e.g., the tip component) or a subsequent third component 103 (e.g., a coupler) or to insulate the first component 101 from the third component 103. In certain examples, the second component 102 can include different features to receive, isolate, position one or more other components in proximity or in contact with different aspects of the lead, such as an electrical contact of the lead, etc.

For example, the second component 102 can include a seal portion 119 to electrically insulate or isolate different portions or components of the stacked bore assembly 100 or a coupled lead, inhibiting fluid communication between different electrical contacts of a lead once the lead is inserted in the bore, providing an interference fit around the inserted lead at the seal portion 119. In an example, the second component 102 can include first and second slots 120, 121 to receive and position a conductive material, such as a conductive beam, in physical and electrical contact with an electrical contact of the inserted lead and, in certain examples, an electrical contact of one or more other components of the stacked bore assembly, such as the third component 103, etc.

In certain examples, the first component 101 can include one or more mechanical features (e.g., a distal mechanical feature) to engage and couple with one or more corresponding mechanical features (e.g., a proximal mechanical feature) of the second component 102. For example, a distal end of the first component 101 can include one of a female (hole) or male (shaft) mating component and the proximal end of the second component 102 can include a corresponding male or female mating component to engage the mating component of the distal end of the first component 101.

In FIG. 1, the distal end of the first component 101 includes a female mating component having a length (e.g., depth) limited by an interior shoulder, illustrated at portion 130. The proximal end of the second component 102 includes a male mating component having a first length from the proximal end of the second component 102 limited by a first exterior shoulder, illustrated at portion 131. The male mating component of the second component can engage the female mating component of the first component 101 up to or near the interior shoulder of the first component 101, illustrated at portion 130, or the first exterior shoulder of the second component 102, illustrated at portion 131.

The distal end of the second component 102 can include a male mating component having a second length from the distal end of the second component 102 limited by a second exterior shoulder, illustrated at portion 132. In certain examples, the second length of the second component 102 is longer than the first length, such as to accommodate the first and second slots 120, 121 and to electrically engage the third component 103 at or near a mid-point of the third component 103. The male mating component at the distal end of the second component 102 can engage a female mating component at a proximal end of the third component 103 (e.g., a coupler) up to or near the second exterior shoulder, illustrated at portion 132, and abut a proximal end of one or more other components (e.g., a proximal end of male mating component of a fourth component 104, such as illustrated in FIG. 1, etc.), illustrated at portion 133.

The third component 103 (e.g., a coupler) can include proximal and distal female mating components, having a length between the proximal and distal ends, configured to engage with and couple successive male mating components of the second component 102, up to the second exterior shoulder of the second component 102, illustrated at portion 132, and one or more other component (e.g., the fourth component 104, the eighth component 108, etc.). In certain examples, the third component 103 can be composed of a conductive material or can include a conductive portion to electrically couple to an electrical contact of the lead, to provide a mating surface for a conductor coupled to a feedthrough of the implantable medical device, etc.

The example of FIG. 1 includes fourth and sixth components 104, 106 substantially similar to the second component 102, and fifth and seventh components 105, 107 substantially similar to the third component 103. In certain examples, the fourth component 104 can include a seal portion 122 and slots 123, 124, and the sixth component 106 can include a seal portion 125 and slots 126, 127, similar to the seal portion 119 and slots 120, 121 of the second component 102, etc. In other examples, more or less core components or couplers can be included.

The fourth component 104 (e.g. a core component) can include proximal and distal male mating components and first and second exterior shoulders illustrated respectively at portions 134, 135. The male mating component at the proximal end of the fourth component 104 can engage the female mating component at the distal end of the third component 103 up to or near the first exterior shoulder of the fourth component 104, illustrated at portion 134. The male mating component at the distal end of the fourth component 104 can engage a female mating component at a proximal end of the fifth component 105 (e.g., a coupler) up to or near the second exterior shoulder, illustrated at portion 135, and abut a proximal end of one or more other components (e.g., a proximal end of male mating component of a sixth component 106, such as illustrated in FIG. 1, etc.), illustrated at portion 136.

The fifth component 105 (e.g., a coupler) can include proximal and distal female mating components, having a length between the proximal and distal ends, configured to engage with and couple successive male mating components of the fourth component 104, up to the second exterior shoulder of the fourth component 104, illustrated at portion 135, and one or more other component (e.g., the sixth component 106, the eighth component 108, etc.). In certain examples, the fifth component 105 can be composed of a conductive material or can include a conductive portion to electrically couple to an electrical contact of the lead, to provide a mating surface for a conductor coupled to a feedthrough of the implantable medical device, etc.

The sixth component 106 (e.g. a core component) can include proximal and distal male mating components and first and second exterior shoulders illustrated respectively at portions 137, 138. The male mating component at the proximal end of the sixth component 106 can engage the female mating component at the distal end of the fifth component 105 up to or near the first exterior shoulder of the sixth component 106, illustrated at portion 137. The male mating component at the distal end of the sixth component 106 can engage a female mating component at a proximal end of the seventh component 107 (e.g., a coupler) up to or near the second exterior shoulder, illustrated at portion 138, and abut a proximal end of one or more other components (e.g., a proximal end of male mating component of a eighth component 108, such as illustrated in FIG. 1, etc.), illustrated at portion 139.

The seventh component 107 (e.g., a coupler) can include proximal and distal female mating components, having a length between the proximal and distal ends, configured to engage with and couple successive male mating components of the sixth component 106, up to the second exterior shoulder of the sixth component 106, illustrated at portion 138, and one or more other component (e.g., the eighth component 108, etc.). In certain examples, the seventh component 107 can be composed of a conductive material or can include a conductive portion to electrically couple to an electrical contact of the lead, to provide a mating surface for a conductor coupled to a feedthrough of the implantable medical device, etc.

The eighth component 108 (e.g., a strain relief component) includes optional steps 109, 110, transitioning to the bore opening 111 of the bore that continues back through the multiple components of the stacked bore assembly 100 to the first component 101. In an example, the eighth component 108 can include a seal portion 128, similar to the seal portion 119 of the second component 102, etc.

The length of the stacked bore assembly 100 can be defined by one or more different measurements, illustrated in FIG. 1 as lengths A1-A6 141-146, between different features of the stacked bore assembly 100. Certain measurements must be controlled within certain tolerances to ensure manufacture and assembly integrity of the stacked bore assembly 100 for incorporation into the header of the implantable medical device. For example, in traditional manufacturing practices, the length (e.g., depth) of the female mating component of the first component 101 would correspond to the length of the male mating component at the proximal end of the second component 102, with each part having positive and negative tolerance. However, worst case scenario might entail situations where tolerances aggregate in one direction, bringing the total assembly out of spec.

In an example, tolerances can be determined and controlled for different measurements. For example, a first plane 147 can be defined as a datum plane for the core and coupler components of the stacked bore assembly 100, between the interior shoulder of the first component 101 and one or more other planes in the stacked bore assembly 100. A second plane 148 can be defined at the distal end of the second component 102, a third plane 149 can be defined at the distal end of the fourth component 104, a fourth plane 150 can be defined at a distal end of the sixth component 106, and a fifth plane 151 can be defined at a distal end of the eighth component 108.

The present inventors have recognized, among other things, techniques to reduce tolerance variation during assembly of different lengths (e.g., A3-A6 143-146) between the different planes, such as by adding strategic clearances by the length or position of different mating features (e.g., interior shoulder, exterior shoulder, length of coupler, etc.) to strategically absorb one side of traditional plus/minus tolerance of or between components. The strategic clearances can be defined, in certain examples, by shoulders or lengths of different mechanical features in contrast to that designed or configured without consideration of positive or negative manufacturing tolerance (e.g., if manufacturing tolerance was ideal, ±0 thou, etc.).

For example, at portion 131, the position of the first exterior shoulder can be designed with a first clearance between the first exterior shoulder of the second component 102 and the distal end (e.g., an edge) of the first component 101 to absorb positive tolerance associated with the length (e.g., depth) of the female mating component of the first component 101 and the length of the first length of the male mating component of the second component 102. In certain examples, the designed distance between the first exterior shoulder of the second component 102 and the proximal end (e.g., an edge) of the second component 102 (e.g., the length of the male mating component at the proximal end of the second component 102) can be purposefully greater than the distance between the interior shoulder of the first component 101 and the distal end of the first component 101, such as by an amount of one or more of the features adding positive or negative tolerance to the female mating component of the first component 101 or the male mating component at the proximal end of the second component 102, etc. For example, the position of the distal end of the first component 101 and the position of the interior shoulder of the first component 101 can each have respective positive and negative tolerances. Similarly, the position of the proximal end of the second component 102 and the position of the first exterior shoulder of the second component 102 can each have respective positive and negative tolerances. A first clearance can be designed with respect to the first exterior shoulder of the second component 102 at portion 131 to absorb tolerance that might otherwise positively impact the overall length of the stacked bore assembly 100, in certain examples, equal to one or more of the positive or negative tolerances impacting the overall length.

In another example, the length of the third component 103, between the proximal and distal ends of the third component 103, can be designed with a second clearance at one or both of portions 132, 134, such that positive tolerance associated with the third component 103 will not positively impact the overall length of the stacked bore assembly 100.

In an example, the distal end of the second component 102 can optionally include a transition of mechanical fits at portion 152, such that mechanical coupling of the second and third components 102, 103 transitions from a slip or transition fit to an interference fit at portion 152, such that during press-fit assembly of the stacked bore assembly 100, compression can stop once the overall length of the stacked bore assembly 100 reaches a desired amount, in certain examples, designing additional clearance to compress into the transition fit at portion 152 with one or more of the clearances described herein. In an example, the length of the interference fit at portion 152 can be greater than the positive or negative tolerance associated with second, third, and fourth components 102-104, such as to absorb positive tolerance or provide additional length to make up for negative tolerance, etc.

In another example, at portion 133, the position of the distal end of the second component 102 or the proximal end of the fourth component 104 can be designed with a third clearance to absorb positive tolerance associated with one or both features.

Similar clearances are shown at subsequent portions of the fourth, fifth, sixth, and seventh components 104-107 as described above. In addition, the distal end of the eighth component 108 can be adjusted (e.g., cut, trimmed, or otherwise reduced) to provide a desired overall length of the stacked bore assembly 100. In certain examples, the different clearances or gaps can be designed, configured, and adjusted such that each of the second, third, fourth, and fifth planes 148-151 can be maintained within specific tolerances with respect to the first plane 147, such that the stacked bore assembly 100 can adhere to tolerances expected from prior single-piece bore assemblies. Accordingly, whereas each of the second through fifth planes 148-151 might have previously had building aggregate tolerances of ±a certain amount (e.g., ±5 thou for the second plane 148, ±10 thou for the third plane 149, etc.), adding gaps and clearances as described herein can limit the tolerances between the negative range and zero, absorbing positive tolerance with the gaps and clearances. In certain examples, the aggregate negative tolerance associated with the multiple components can be added to the length of the eighth component 108 and adjusted (e.g. reduced) during assembly if needed. However, during assembly, it is more likely that the stack will not be fully pressed than for aggregate negative tolerances to impact the overall length of the stacked bore assembly 100 out of spec.

In certain examples, the multiple components can be press fit in a single step. In other examples, the core components (and one or more couplers, such as all couplers or all but the last coupled to the strain relief segment) can be press fit to the tip component in a single step, with the strain relief segment (and in certain examples, the last coupler between the last core component and the strain relief segment) in a second step, such as to ensure full contact between the core components while pressing the last component or components in a second step to control a desired overall length (selectively not fully press the strain relief segment to extend the length of the assembly in case of aggregate negative tolerances, etc.).

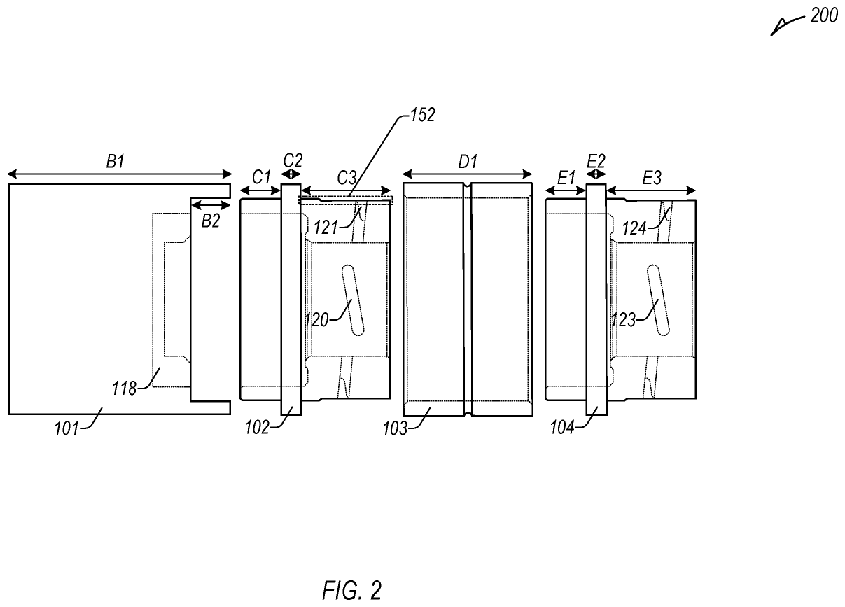

FIG. 2 illustrates a first subset of components 200 including separate first, second, third, and fourth components 101-104 prior to being press fit to assemble the stacked bore assembly 100 illustrated in FIG. 1, or a portion thereof.

The first component 101 has a first length B1 between proximal and distal ends of the first component 101, and a second length B2 commensurate with the length (e.g., depth) of the female mating component at the distal end of the first component 101, from the distal end of the first component 101 to the interior shoulder, such as illustrated at portion 130 in FIG. 1.

The second component 102 has a total length as a combination of a first length C1 between a proximal end of the second component 102 and the first exterior shoulder of the second component 102, illustrated at portion 131 in FIG. 1, a second length C2 between the first exterior shoulder and the second exterior shoulder of the second component 102, illustrated at portion 132 in FIG. 1, and a third length C3 between a distal end of the second component 102 and the second exterior shoulder of the second component 102.

The third component 103 has a length D1 between proximal and distal ends of the third component 103, and the fourth component 104 has a total length as a combination of first, second, and third lengths E1-E3, similar to those described with respect to the second component 102.

FIG. 3 illustrates a second subset of components 300 including separate fifth, sixth, seventh, and eight components 105-108 prior to being press fit to assemble the stacked bore assemble 100 illustrated in FIG. 1, or a portion thereof.

The fifth and seventh components 105, 107 have lengths F1 and H1, respectively, between proximal and distal ends of the respective fifth and seventh components 105, 107. The sixth component 106 has a total length as a combination of first, second, and third lengths G1-G3, similar to those described with respect to the second component 102 in FIG. 2.

The eighth component 108 has a total length as a combination of a first length I1 between a proximal end of the eighth component 108 and a first exterior shoulder of the eighth component 108, a second length I2 between the first exterior shoulder and a second exterior shoulder of the eighth component 108, and a third length I3 between a distal end of the eighth component 108 and the second exterior shoulder of the eighth component 108, including steps 109 and 110.

With respect to the lengths in FIGS. 2-3, gaps or clearances commensurate to positive tolerances of one or more components can be added to different ones of the different lengths described above in different permutations or combinations to accomplish different objectives. For example, adding a clearance to C1 (moving the exterior shoulder into C2, effectively reducing C2) can absorb positive tolerance associated with different aspects of the first or second components 101, 102. Reducing a length of C1 (from the proximal end of the second component 102) can absorb positive tolerance associated with different aspects of the first and second components 101, 102. Adding a clearance to C3 (moving the exterior shoulder into C2, effectively reducing C2) can absorb positive tolerance associated with different aspects of the second or third components 102, 103. Reducing a length of C3 (from the distal end of the second component 102) can absorb positive tolerance associated with one of the second or fourth components 102, 104. Adding a clearance to D1, such as by effectively reducing the length of D1, can absorb positive tolerance associated with different aspects of the second, third, or fourth components 102-104. The transition at portion 152 can provide for additional length, such as by not fully pressing the second, third, or fourth components 102-104 together in the case of negative tolerance. Similar gaps, clearances, or adjustments can be made to the fourth component 104, or the fifth through seventh components 105-107.

With respect to the eighth component 108, adding a clearance to I1, effectively reducing I2, can absorb positive tolerance associated with different aspects of the seventh or eighth components 107, 108. Reducing a length of I1 can absorb positive tolerance associated with different aspects of the seventh or eighth components 107, 108. In contrast, adding length to one or both of I2 or I3 can compensate for negative clearances associated with one or more of the multiple components of the stack. In other examples, different components can be pressed together as needed in one or more steps prior to placing the stacked bore assembly 100 in position to be formed in the header, such as by one or more manufacturing processes (e.g., overmolding, etc.), to more distinctly control the different lengths and planes of the stacked bore assembly 100.

Although illustrated herein in FIGS. 1-3 with the first component 101 having a female mechanical joining feature, the second component 102 having two male mechanical joining features, the third component 103 having two female mechanical joining features, etc., in practice such features can be switched or changed, so long as the tip and core components are able to mechanically couple to each other. In other examples, the female and male mechanical joining features described herein can be switched, or instead of two male (or female) mechanical joining features on proximal and distal ends, the core components and the coupler can have one male mechanical joining feature and one female mechanical joining feature, or other permutations or combinations.

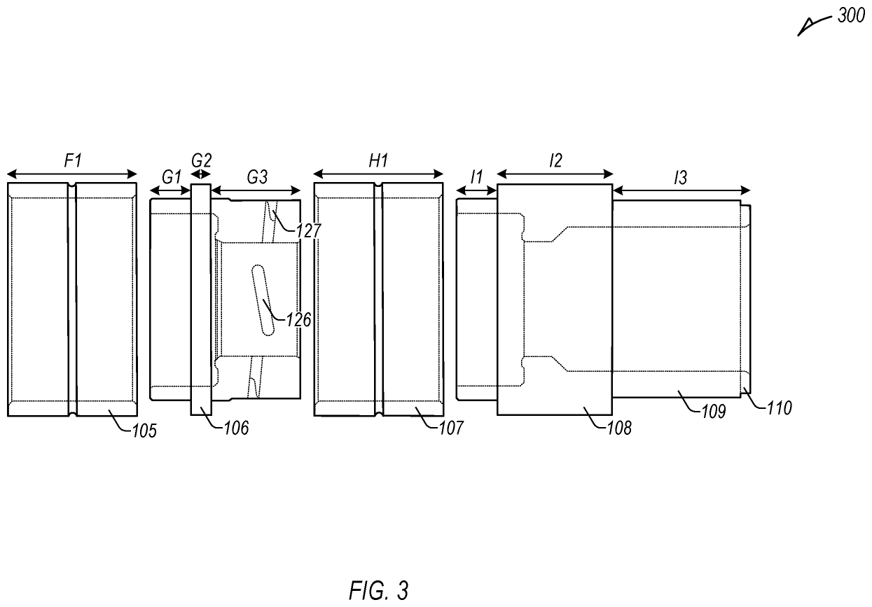

FIG. 4 illustrates an example implantable medical device 400 including a hermetically sealed housing 401, a header 402 comprising first and second stacked bore assemblies 403, 404, a feedthrough 405 including one or more feedthrough pins or connections, and first, second, third, and fourth wires 406-409 configured to couple to different contacts of the first stacked bore assembly 403.

The first wire 406 is configured to couple to a conductive surface or contact of a tip component of the first stacked bore assembly 403. The second, third, and fourth wires 407-409 are configured to couple to respective conductive surfaces or contacts of respective first, second, and third couplers of the stacked bore assembly 403. Although illustrated as stopping at the bottom surface of the respective components in FIG. 4, in practice, the wires can proceed to contact the bottom or a side surface of such components, in various combinations or permutations.

Additional wires (not illustrated) can be routed from other feedthrough pins or connections of the feedthrough 405 to different contacts of second stacked bore assembly 404. In other examples, one or more additional components can be coupled to one or more of the feedthrough pins or connections, such as an antenna, etc. Further, in other examples, the feedthrough 405 can be located on a top portion of the housing 401, one or more other sides of the housing, or various permutations or combinations, etc.

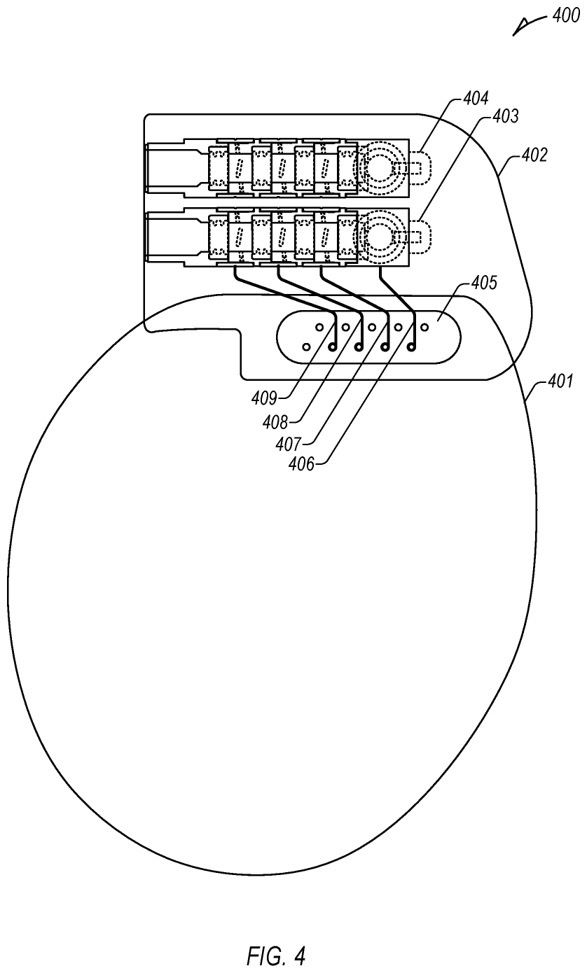

FIG. 5 illustrates an example medical device system 500, such as an implantable medical device, a cardiac rhythm management (CRM) device, etc. In an example, one or more aspects of the example system 500 can be a component of, or communicatively coupled to, an implantable medical device, an insertable cardiac monitor, etc. The system 500 can be configured to monitor, detect, or treat various physiologic conditions of the body, such as cardiac conditions associated with a reduced ability of a heart to sufficiently deliver blood to a body, including heart failure, arrhythmias, dyssynchrony, etc., or one or more other physiologic conditions and, in certain examples, can be configured to provide electrical stimulation or one or more other therapies or treatments to the patient.

The system 500 can include a single medical device or a plurality of medical devices implanted in a body of a patient or otherwise positioned on or about the patient to monitor patient physiologic information of the patient using one or more sensors, such as a sensor 501. In an example, the sensor 501 can include one or more of: a respiration sensor configured to receive respiration information (e.g., a respiration rate, a respiration volume (tidal volume), etc.); an acceleration sensor (e.g., an accelerometer, a microphone, etc.) configured to receive cardiac acceleration information (e.g., cardiac vibration information, pressure waveform information, heart sound information, endocardial acceleration information, acceleration information, activity information, posture information, etc.); an impedance sensor (e.g., intrathoracic impedance sensor, transthoracic impedance sensor, etc.) configured to receive impedance information, a cardiac sensor configured to receive cardiac electrical information; an activity sensor configured to receive information about a physical motion (e.g., activity, steps, etc.); a posture sensor configured to receive posture or position information; a pressure sensor configured to receive pressure information; a plethysmograph sensor (e.g., a photoplethysmography sensor, etc.); a chemical sensor (e.g., an electrolyte sensor, a pH sensor, an anion gap sensor, etc.); a temperature sensor; a skin elasticity sensor, or one or more other sensors configured to receive physiologic information of the patient.

The example system 500 can include a signal receiver circuit 502 and an assessment circuit 503. The signal receiver circuit 502 can be configured to receive physiologic information of a patient (or group of patients) from the sensor 501. The assessment circuit 503 can be configured to receive information from the signal receiver circuit 502, and to determine one or more parameters (e.g., physiologic parameters, stratifiers, etc.) or existing or changed patient conditions using the received physiologic information, such as described herein. The physiologic information can include, among other things, cardiac electrical information, impedance information, respiration information, heart sound information, activity information, posture information, temperature information, or one or more other types of physiologic information.

In certain examples, the assessment circuit 503 can aggregate information from multiple sensors or devices, detect various events using information from each sensor or device separately or in combination, update a detection status for one or more patients based on the information, and transmit a message or an alert to one or more remote devices that a detection for the one or more patients has been made or that information has been stored or transmitted, such that one or more additional processes or systems can use the stored or transmitted detection or information for one or more other review or processes.

The assessment circuit 503 can be configured to provide an output to a user, such as to a display or one or more other user interface, the output including a score, a trend, an alert, or other indication. In other examples, the assessment circuit 503 can be configured to provide an output to another circuit, machine, or process, such as a therapy circuit 504 (e.g., a cardiac resynchronization therapy (CRT) circuit, a chemical therapy circuit, etc.), etc., to control, adjust, or cease a therapy of a medical device, a drug delivery system, etc., or otherwise alter one or more processes or functions of one or more other aspects of a medical-device system, such as one or more cardiac resynchronization therapy parameters, drug delivery, dosage determinations or recommendations, etc. In an example, the therapy circuit 504 can include one or more of a stimulation control circuit, a cardiac stimulation circuit, a neural stimulation circuit, a dosage determination or control circuit, etc. In other examples, the therapy circuit 504 can be controlled by the assessment circuit 503, or one or more other circuits, etc.

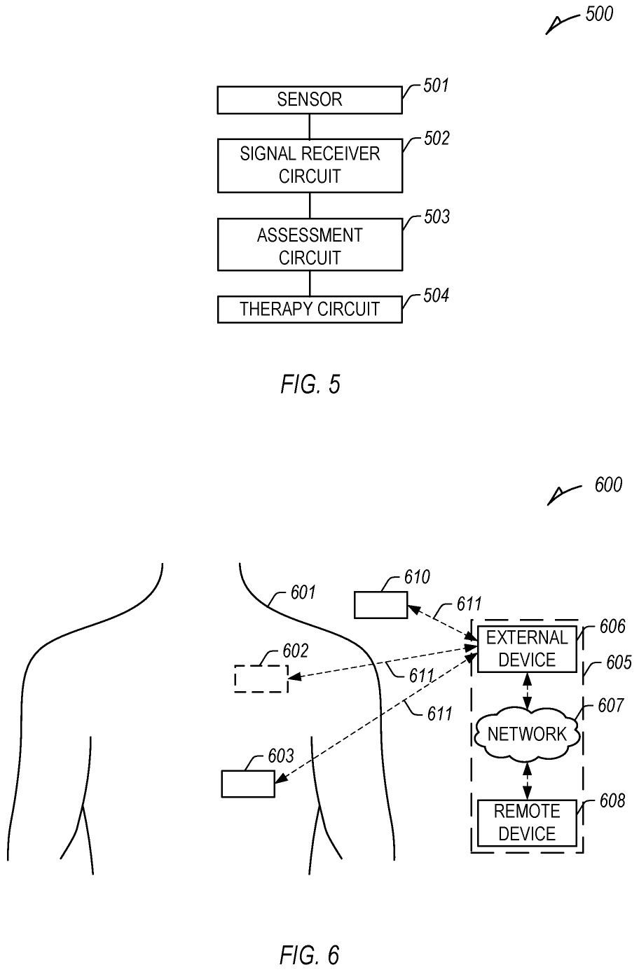

FIG. 6 illustrates an example patient management system 600 and portions of an environment in which the patient management system 600 may operate. The patient management system 600 can perform a range of activities, including remote patient monitoring and diagnosis of a disease condition. Such activities can be performed close to or near a patient 601, such as in a patient home or office, through a centralized server, such as in a hospital, clinic, or physician office, or through a remote workstation, such as a secure wireless mobile computing device.

The patient management system 600 can include one or more ambulatory medical devices, an external system 605, and a communication link 611 providing for communication between the one or more ambulatory medical devices and the external system 605. The one or more ambulatory medical devices can include an implantable medical device (IMD) 602, a wearable medical device 603, or one or more other implantable, leadless, subcutaneous, external, wearable, or ambulatory medical devices configured to monitor, sense, or detect information from, determine physiologic information about, or provide one or more therapies to treat various conditions of the patient 601, such as one or more cardiac or non-cardiac conditions (e.g., dehydration, sleep disordered breathing, etc.).

In an example, the implantable medical device 602 can include one or more traditional cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 601. In another example, the implantable medical device 602 can include a monitor implanted, for example, subcutaneously in the chest of patient 601, the implantable medical device 602 including a housing containing circuitry and, in certain examples, one or more sensors, such as a temperature sensor, etc.

Traditional cardiac rhythm management devices, such as insertable cardiac monitors, pacemakers, defibrillators, or cardiac resynchronizers, include implantable or subcutaneous devices having hermetically sealed housings configured to be implanted in a chest of a patient. The cardiac rhythm management device can include one or more leads to position one or more electrodes or other sensors at various locations in or near the heart, such as in one or more of the atria or ventricles of a heart, etc. Accordingly, cardiac rhythm management devices can include aspects located subcutaneously, though proximal to the distal skin of the patient, as well as aspects, such as leads or electrodes, located near one or more organs of the patient. Separate from, or in addition to, the one or more electrodes or other sensors of the leads, the cardiac rhythm management device can include one or more electrodes or other sensors (e.g., a pressure sensor, an accelerometer, a gyroscope, a microphone, etc.) powered by a power source in the cardiac rhythm management device. The one or more electrodes or other sensors of the leads, the cardiac rhythm management device, or a combination thereof, can be configured detect physiologic information from the patient, or provide one or more therapies or stimulation to the patient.

Implantable devices can additionally or separately include leadless cardiac pacemakers (LCPs), small (e.g., smaller than traditional implantable cardiac rhythm management devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable cardiac rhythm management device complications (e.g., required incision and pocket, complications associated with lead placement, breakage, or migration, etc.). In certain examples, leadless cardiac pacemakers can have more limited power and processing capabilities than a traditional cardiac rhythm management device; however, multiple leadless cardiac pacemakers can be implanted in or about the heart to detect physiologic information from, or provide one or more therapies or stimulation to, one or more chambers of the heart. The multiple leadless cardiac pacemaker can communicate between themselves, or one or more other implanted or external devices.

The implantable medical device 602 can include an assessment circuit configured to detect or determine specific physiologic information of the patient 601, or to determine one or more conditions or provide information or an alert to a user, such as the patient 601 (e.g., a patient), a clinician, or one or more other caregivers or processes, such as described herein. The implantable medical device 602 can alternatively or additionally be configured as a therapeutic device configured to treat one or more medical conditions of the patient 601. The therapy can be delivered to the patient 601 via the lead system and associated electrodes or using one or more other delivery mechanisms. The therapy can include delivery of one or more drugs to the patient 601, such as using the implantable medical device 602 or one or more of the other ambulatory medical devices, etc. In some examples, therapy can include cardiac resynchronization therapy for rectifying dyssynchrony and improving cardiac function in heart failure patients. In other examples, the implantable medical device 602 can include a drug delivery system, such as a drug infusion pump to deliver drugs to the patient for managing arrhythmias or complications from arrhythmias, hypertension, hypotension, or one or more other physiologic conditions. In other examples, the implantable medical device 602 can include one or more electrodes configured to stimulate the nervous system of the patient or to provide stimulation to the muscles of the patient airway, etc.

The wearable medical device 603 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

The external system 605 can include a dedicated hardware/software system, such as a programmer, a remote server-based patient management system, or alternatively a system defined predominantly by software running on a standard personal computer. The external system 605 can manage the patient 601 through the implantable medical device 602 or one or more other ambulatory medical devices connected to the external system 605 via a communication link 611. In other examples, the implantable medical device 602 can be connected to the wearable medical device 603, or the wearable medical device 603 can be connected to the external system 605, via the communication link 611. This can include, for example, programming the implantable medical device 602 to perform one or more of acquiring physiologic data, performing at least one self-diagnostic test (such as for a device operational status), analyzing the physiologic data, or optionally delivering or adjusting a therapy for the patient 601. Additionally, the external system 605 can send information to, or receive information from, the implantable medical device 602 or the wearable medical device 603 via the communication link 611. Examples of the information can include real-time or stored physiologic data from the patient 601, diagnostic data, such as detection of patient hydration status, hospitalizations, responses to therapies delivered to the patient 601, or device operational status of the implantable medical device 602 or the wearable medical device 603 (e.g., battery status, lead impedance, etc.). The communication link 611 can be an inductive telemetry link, a capacitive telemetry link, or a radio-frequency (RF) telemetry link, or wireless telemetry based on, for example, “strong” Bluetooth or IEEE 602.11 wireless fidelity “Wi-Fi” interfacing standards. Other configurations and combinations of patient data source interfacing are possible.

The external system 605 can include an external device 606 in proximity of the one or more ambulatory medical devices, and a remote device 608 in a location relatively distant from the one or more ambulatory medical devices, in communication with the external device 606 via a communication network 607. Examples of the external device 606 can include a medical device programmer. The remote device 608 can be configured to evaluate collected patient or patient information and provide alert notifications, among other possible functions. In an example, the remote device 608 can include a centralized server acting as a central hub for collected data storage and analysis from a number of different sources. Combinations of information from the multiple sources can be used to make determinations and update individual patient status or to adjust one or more alerts or determinations for one or more other patients. The server can be configured as a uni-, multi-, or distributed computing and processing system. The remote device 608 can receive data from multiple patients. The data can be collected by the one or more ambulatory medical devices, among other data acquisition sensors or devices associated with the patient 601. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

The remote device 608 may additionally include one or more locally configured clients or remote clients securely connected over the communication network 607 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 608, including the server and the interconnected clients, may also execute a follow-up scheme by sending follow-up requests to the one or more ambulatory medical devices, or by sending a message or other communication to the patient 601 (e.g., the patient), clinician or authorized third party as a compliance notification.

The communication network 607 can provide wired or wireless interconnectivity. In an example, the communication network 607 can be based on the Transmission Control Protocol/Internet Protocol (TCP/IP) network communication specification, although other types or combinations of networking implementations are possible. Similarly, other network topologies and arrangements are possible.

One or more of the external device 606 or the remote device 608 can output the detected medical events to a system user, such as the patient or a clinician, or to a process including, for example, an instance of a computer program executable in a microprocessor. In an example, the process can include an automated generation of recommendations for anti-arrhythmic therapy, or a recommendation for further diagnostic test or treatment. In an example, the external device 606 or the remote device 608 can include a respective display unit for displaying the physiologic or functional signals, or alerts, alarms, emergency calls, or other forms of warnings to signal the detection of arrhythmias. In some examples, the external system 605 can include an external data processor configured to analyze the physiologic or functional signals received by the one or more ambulatory medical devices, and to confirm or reject the detection of arrhythmias. Computationally intensive algorithms, such as machine-learning algorithms, can be implemented in the external data processor to process the data retrospectively to detect cardia arrhythmias.

Portions of the one or more ambulatory medical devices or the external system 605 can be implemented using hardware, software, firmware, or combinations thereof. Portions of the one or more ambulatory medical devices or the external system 605 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

The patient management system 600 can include a therapy device 610, such as ambulatory or external therapy device configured to send information to or receive information from one or more of the ambulatory medical devices or the external system 605 using the communication link 611. In an example, the one or more ambulatory medical devices, the external device 606, or the remote device 608 can be configured to control one or more parameters of the therapy device 610. The external system 605 can allow for programming the one or more ambulatory medical devices and can receives information about one or more signals acquired by the one or more ambulatory medical devices, such as can be received via a communication link 611. The external system 605 can include a local external implantable medical device programmer. The external system 605 can include a remote patient management system that can monitor patient status or adjust one or more therapies such as from a remote location.

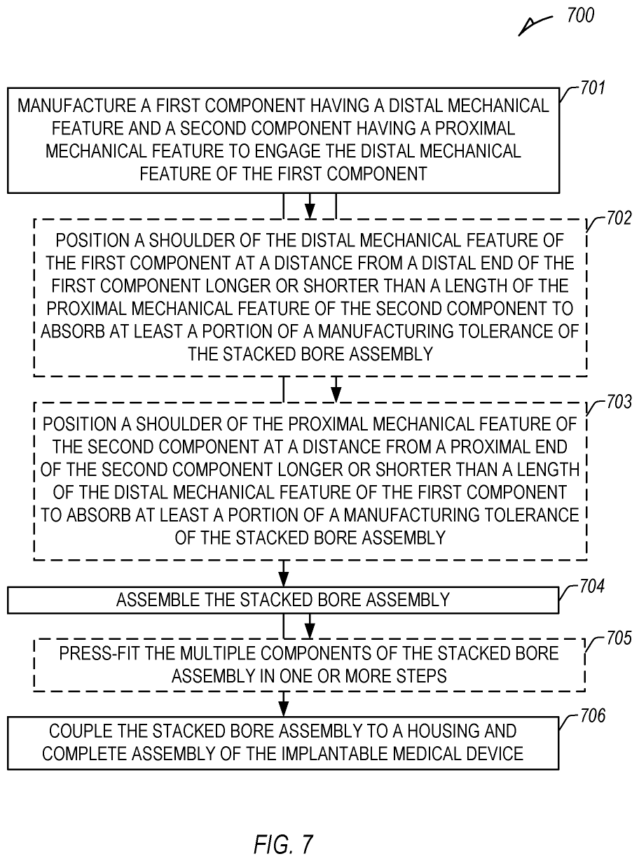

FIG. 7 illustrates an example method 700 of assembling a stacked bore assembly and subsequently assembling an implantable medical device.

At step 701, different components of the stacked bore assembly are manufactured, each including respective mechanical features. The different components can include a tip component, one or more core components, one or more couplers, and a strain relief segment, such as otherwise described herein. The first component can include a distal mechanical feature, and the second component can include a proximal mechanical feature configured to engage the distal mechanical feature of the first component.

At step 702, a shoulder of the distal mechanical feature of the first component can optionally be positioned at a distance from a distal end of the first component longer or shorter than a length of the proximal mechanical feature of the second component to absorb at least a portion of a manufacturing tolerance of one or more components of the stacked bore assembly, such as one or more features of the first or second components, one or more components of the distal mechanical feature of the first component or the proximal mechanical feature of the second component, etc. For example, positioning the shoulder of the distal mechanical feature of the first component can include adding a clearance or a gap to absorb a positive manufacturing tolerance associated with the length of the proximal mechanical feature of the second component, or the length of the distal mechanical feature the first component itself, such that positive tolerance associated with such component will not add to overall stack length of the assembled stacked bore assembly. In other examples, one or more other clearances or gaps or lengths can be added to one or more components to further reduce one or both of positive or negative manufacturing tolerance of the stacked bore assembly.

At step 703, a shoulder of the proximal mechanical feature of the second component can optionally be positioned at a distance from a proximal end of the second component longer or shorter than a length of the distal mechanical feature of the first component to absorb at least a portion of a manufacturing tolerance of one or more components of the stacked bore assembly, such as one or more features of the first or second components, one or more components of the distal mechanical feature the first component or the proximal mechanical feature of the second component, etc. For example, positioning the shoulder of the proximal mechanical feature of the second component can include adding a clearance or a gap to absorb a positive manufacturing tolerance associated with the length of the distal mechanical feature of the first component, or the length of the proximal mechanical feature of the second component itself, such that positive tolerance associated with such component will not add to overall stack length of the assembled stacked bore assembly.

At step 704, the stacked bore assembly can be assembled, including at step 705, by optionally press-fitting the multiple components of the stacked bore assembly, such as described herein, in one or more steps. For example, the first and second components can be placed near each other and press fit in a first step, and the other components can be engaged with the first and second components in one or more additional steps. In one example, each component can be press-fit in separate steps with measurement to ensure assembly withing strict manufacturing tolerances. In another example, all major components of the stacked bore assembly, such as first through eighth components 101-108, etc., can be assembled in a single press-fitting step, such as using a machine or process. In certain examples, one or more of the overall stack length or different planes of the stacked bore assembly can be measured during or after assembly of the stacked bore assembly.

At step 706, the stacked bore assembly, once completed, can be coupled to a housing, such as by connecting one or more electrical contacts of the stacked bore assembly to one or more feedthrough connections on a housing of an implantable medical device. Following connection of the stacked bore assembly to the housing, assembly of the implantable medical device can be completed, such as by forming the header, including epoxying the multiple components of the header to protect the different components and the subject and overmolding the header to the implantable medical device.

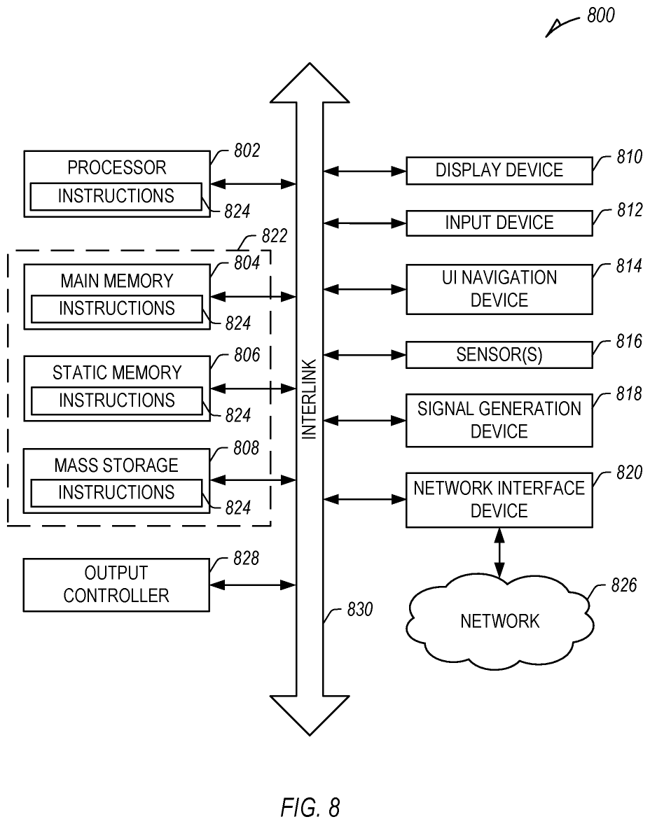

FIG. 8 illustrates a block diagram of an example machine 800 upon which any one or more of the techniques (e.g., methodologies) discussed herein may perform. Portions of this description may apply to the computing framework of one or more of the medical devices described herein, such as the implantable medical device, the external programmer, etc. Further, as described herein with respect to medical device components, systems, or machines, such may require regulatory-compliance not capable by generic computers, components, or machinery.

Examples, as described herein, may include, or may operate by, logic or a number of components, or mechanisms in the machine 800. Circuitry (e.g., processing circuitry, an assessment circuit, etc.) is a collection of circuits implemented in tangible entities of the machine 800 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 800 follow.

In alternative embodiments, the machine 800 may operate as a standalone device or may be connected (e.g., networked) to other machines. In a networked deployment, the machine 800 may operate in the capacity of a server machine, a client machine, or both in server-client network environments. In an example, the machine 800 may act as a peer machine in peer-to-peer (P2P) (or other distributed) network environment. The machine 800 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine (e.g., computer system) 800 may include a hardware processor 802 (e.g., a central processing unit (CPU), a graphics processing unit (GPU), a hardware processor core, or any combination thereof), a main memory 804, a static memory (e.g., memory or storage for firmware, microcode, a basic-input-output (BIOS), unified extensible firmware interface (UEFI), etc.) 806, and mass storage 808 (e.g., hard drive, tape drive, flash storage, or other block devices) some or all of which may communicate with each other via an interlink (e.g., bus) 830. The machine 800 may further include a display unit 810, an input device 812 (e.g., a keyboard), and a user interface (UI) navigation device 814 (e.g., a mouse). In an example, the display unit 810, input device 812, and UI navigation device 814 may be a touch screen display. The machine 800 may additionally include a signal generation device 818 (e.g., a speaker), a network interface device 820, and one or more sensors 816, such as a global positioning system (GPS) sensor, compass, accelerometer, or one or more other sensors. The machine 800 may include an output controller 828, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the hardware processor 802, the main memory 804, the static memory 806, or the mass storage 808 may be, or include, a machine-readable medium 822 on which is stored one or more sets of data structures or instructions 824 (e.g., software) embodying or utilized by any one or more of the techniques or functions described herein. The instructions 824 may also reside, completely or at least partially, within any of registers of the hardware processor 802, the main memory 804, the static memory 806, or the mass storage 808 during execution thereof by the machine 800. In an example, one or any combination of the hardware processor 802, the main memory 804, the static memory 806, or the mass storage 808 may constitute the machine-readable medium 822. While the machine-readable medium 822 is illustrated as a single medium, the term “machine-readable medium” may include a single medium or multiple media (e.g., a centralized or distributed database, and/or associated caches and servers) configured to store the one or more instructions 824.

The term “machine-readable medium” may include any medium that is capable of storing, encoding, or carrying instructions for execution by the machine 800 and that cause the machine 800 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 824 may be further transmitted or received over a communications network 826 using a transmission medium via the network interface device 820 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 820 may include one or more physical jacks (e.g., Ethernet, coaxial, or phone jacks) or one or more antennas to connect to the communications network 826. In an example, the network interface device 820 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 800, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A system, comprising:
a stacked bore assembly comprising multiple components between proximal and distal ends of the stacked bore assembly, the stacked bore assembly configured to receive a lead in a bore of the stacked bore assembly and to electrically couple to one or more electrical contacts on the lead once the lead is inserted into and secured in the bore of the stacked bore assembly, the stacked bore assembly comprising:
a first component comprising a distal mechanical feature; and
a second component comprising a proximal mechanical feature configured to engage the distal mechanical feature of the first component to a shoulder on a first one of the distal mechanical feature of the first component or the proximal mechanical feature of the second component,
wherein the shoulder is positioned at a distance from an end of the first one of the distal mechanical feature of the first component or the proximal mechanical feature of the second component shorter or longer than a length of a second of the distal mechanical feature of the first component or the proximal mechanical feature of the second component to absorb a portion of a positive or negative manufacturing tolerance of at least one of the first component or the second component.

2. The system of claim 1, wherein the stacked bore assembly is a component of a header of an implantable medical device,
wherein the implantable medical device comprises a housing comprising electronic circuitry,
wherein the first component comprises a first electrical contact to couple a first electronic circuit of the electronic circuitry of the housing of the implantable medical device to the first electrical contact of the stacked bore assembly,
wherein the second component comprises a second electrical contact to couple a second electronic circuit of the electronic circuitry of the housing of the implantable medical device to the second electrical contact of the stacked bore assembly,
wherein the stacked bore assembly is configured to receive a lead comprising first and second electrical contacts, wherein the first electrical contact of the lead, when inserted into and retained by the stacked bore assembly, is configured to couple to the first electrical contact of the first component of the stacked bore assembly, and wherein the second electrical contact of the lead, when inserted into and retained by the stacked bore assembly, is configured to couple to the second electrical contact of the second component of the stacked bore assembly.

3. The system of claim 1, wherein the distal mechanical feature of the first component comprises a male mechanical feature,
wherein the proximal mechanical feature of the second component comprises a female mechanical feature configured to interference fit with the male mechanical feature to the shoulder on one of the male mechanical feature or the female mechanical feature.

4. The system of claim 1, wherein the distal mechanical feature of the first component comprises a female mechanical feature,
wherein the proximal mechanical feature of the second component comprises a male mechanical feature configured to interference fit with the female mechanical feature to the shoulder on one of the male mechanical feature or the female mechanical feature.

5. The system of claim 1, wherein the second component comprises a distal mechanical feature,
wherein the stacked bore assembly comprises:
a third component comprising a coupler having proximal and distal mechanical features; and
a fourth component comprising a proximal mechanical feature,
wherein the coupler is configured to engage the distal mechanical feature of the second component and the proximal mechanical feature of the fourth component between respective shoulders of the second component and the fourth component, the coupler having a length shorter or longer than a corresponding distance between the respective shoulders when the fourth component is joined with the second component by the third component to absorb a portion of one of a positive or negative manufacturing tolerance of at least one of the second component, the third component, or the fourth component.

6. The system of claim 5, wherein the coupler comprises a conductive material configured to be electrically connected to a second electrical contact of the second component when the coupler is engaged with the second component,
wherein the second component comprises a core component composed of an insulator material including an interior mechanical feature to position the second electrical contact of the second component with an electrical contact of the lead when inserted into and retained by the stacked bore assembly, wherein the insulator material of the second component is configured to electrically insulate the conductive material of the coupler from a first electrical contact of the first component,
wherein the fourth component comprises a core component substantially similar to the second component.

7. The system of claim 6, wherein the first component comprises a tip component at a proximal end of the stacked bore assembly, the tip component configured to receive and retain a proximal end of a lead once inserted into the stacked bore assembly,
wherein the stacked bore assembly comprises multiple core components joined by multiple couplers between the tip component and a distal end of the stacked bore assembly.

8. The system of claim 5, wherein the distal mechanical feature of the second component comprises a transition of mechanical fits with respect to a proximal end of the third component, from a slip or transition fit with the proximal mechanical feature of the third component when the third component is engaged with the second component at a distal end of the distal mechanical feature to an interference fit at a proximal end of the distal mechanical feature, wherein the length of the interference fit is greater than a positive or negative manufacturing tolerance of at least one of the second component or the third component.

9. The system of claim 1, wherein the shoulder is positioned at the distance from an edge of the distal mechanical feature of the first component shorter than the length of the proximal mechanical feature of the second component to absorb a portion of the positive manufacturing tolerance of at least one of the first component or the second component.

10. The system of claim 1, wherein the shoulder includes:
a first interior shoulder on a female mating component of a distal end of the first component; and
a second exterior shoulder on a male mating component of a proximal end of the second component,
wherein the first interior shoulder on the female mating component of the distal end of the first component is positioned at a distance shorter than a length of the male mating component of the proximal end of the second component to absorb a portion of a positive or negative manufacturing tolerance of at least one of the first component or the second component,
wherein the length of the male mating component of the proximal end of the second component comprises a distance from the proximal end of the second component to the second exterior shoulder on the male mating component of the proximal end of the second component.

11. The system of claim 1, wherein the shoulder includes:
a first interior shoulder on a female mating component of a distal end of the first component; and
a second exterior shoulder on a male mating component of a proximal end of the second component,
wherein the second exterior shoulder on the male mating component of the proximal end of the second component is positioned at a distance longer than a length of the female mating component of the distal end of the first component from the proximal end of the second component to absorb a portion of a positive or negative manufacturing tolerance of at least one of the first component or the second component,
wherein the length of the female mating component of the distal end of the first component comprises a distance from the distal end of the first component to the first interior shoulder on the female mating component of the distal end of the first component.

12. The system of claim 1, wherein the distance of the position of the second exterior shoulder from proximal end of the second component comprises an aggregate positive or negative manufacturing tolerance of at least one first or second components with respect to a plane defined by one of the first component or the second component.

13. The system of claim 1, wherein the distance of the position of the second exterior shoulder from proximal end of the second component comprises the positive or negative manufacturing tolerance of at least one the female mating component of the distal end of the first component or the male mating component of the proximal end of the second component.

14. A system, comprising:
a stacked bore assembly comprising multiple components, the stacked bore assembly configured to receive a lead in a bore of the stacked bore assembly and to electrically couple to one or more electrical contacts on the lead once the lead is inserted into and secured in the bore of the stacked bore assembly, the stacked bore assembly comprising:
a first component comprising a distal mechanical feature; and
a second component comprising a proximal mechanical feature configured to engage the distal mechanical feature of the first component,
wherein the distal mechanical feature of the first component is configured to engage the proximal mechanical feature of the second component to a shoulder on the proximal mechanical feature of the second component,
wherein the shoulder is positioned at a distance from a proximal end of the second component longer than a length of the distal mechanical feature of the first component to absorb a portion of one of a positive or negative manufacturing tolerance of at least one of the first component or the second component.

15. The system of claim 14, wherein the second component comprises a distal mechanical feature,
wherein the stacked bore assembly comprises:
a third component comprising a coupler to engage a proximal mechanical feature of a fourth component and the distal mechanical feature of the second component between respective shoulders of the second component and the fourth component, the coupler having a length shorter or longer than a corresponding distance between the respective shoulders when the fourth component is joined with the second component by the third component to absorb a portion of one of a positive or negative manufacturing tolerance of at least one of the second component, the third component, or the fourth component.

16. The system of claim 15, wherein the first component and the third component include conductive material to couple to respective feedthrough connections of a housing of an implantable medical device,
wherein the second component includes an insulator to insulate the conductive material of the first component from the conductive material of the third component.

17. The system of claim 15, wherein the first component comprises a tip component of the stacked bore assembly,
wherein the second component and the fourth component comprise core components of the stacked bore assembly,
wherein the third component comprises a coupler,
wherein the stacked bore assembly comprises multiple core components and multiple couplers between proximal and distal ends of the stacked bore assembly, each coupler and core component substantially similar to the other respective couplers or core components.

18. The system of claim 17, comprising a strain relief component at a distal end of the stacked bore assembly, opposite the tip component.

19. A system, comprising:
a stacked bore assembly comprising multiple components, the stacked bore assembly configured to receive a lead in a bore of the stacked bore assembly and to electrically couple to one or more electrical contacts on the lead once the lead is inserted into and secured in the bore of the stacked bore assembly, the stacked bore assembly comprising:
a first component comprising a distal mechanical feature; and
a second component comprising a proximal mechanical feature configured to engage the distal mechanical feature of the first component to a shoulder on the distal mechanical feature of the first component,
wherein the shoulder is positioned at a distance from a distal end of the first component longer than a length of the proximal mechanical feature of the second component to absorb a portion of one of a positive or negative manufacturing tolerance of at least one of the first component or the second component.

20. The system of claim 19, wherein the second component comprises a distal mechanical feature,
wherein the stacked bore assembly comprises:
a third component comprising a coupler having proximal and distal mechanical features; and
a fourth component comprising a proximal mechanical feature,
wherein the coupler is configured to engage the distal mechanical feature of the second component and the proximal mechanical feature of the fourth component between respective shoulders of the second component and the fourth component, the coupler having a length shorter or longer than a corresponding distance between the respective shoulders when the fourth component is joined with the second component by the third component to absorb a portion of one of a positive or negative manufacturing tolerance of at least one of the second component, the third component, or the fourth component,
wherein the distal mechanical feature of the second component comprises a transition of mechanical fits with respect to a proximal end of the third component, from a slip or transition fit with the proximal mechanical feature of the third component when the third component is engaged with the second component at a distal end of the distal mechanical feature to an interference fit at a proximal end of the distal mechanical feature, wherein the length of the interference fit is greater than a positive or negative manufacturing tolerance of at least one of the second component or the third component.
